(19) United States
(12) Patent Application Publication
(10) Pub. No.: US 2016/0355980 A1
(43) Pub. Date: Dec. 8, 2016

(54) Absorbent Fibrous Structures Comprising a Branched Copolymer Soil Adsorbing Agent

(71) Applicant: The Procter & Gamble Company, Cincinnati, OH (US)

(72) Inventors: Kathryn Christian Kien, Cincinnati, OH (US); Robin Lynn McKiernan, Mason, OH (US); Steven Daryl Smith, Fairfield, OH (US); Robert Joseph McChain, Cincinnati, OH (US)

(21) Appl. No.: 15/170,985

(22) Filed: Jun. 2, 2016

Related U.S. Application Data
(60) Provisional application No. 62/169,770, filed on Jun. 2, 2015.

(51) Int. Cl. D21H 21/22; D21H 27/30; A61F 13/15; A61L 15/24; A61L 15/40; A61L 15/60; A61L 15/48; D21H 27/00; A61F 13/53

(52) U.S. Cl. CPC D21H 21/22; D21H 27/004; D21H 27/30; A61F 13/15203; A61F 13/53; A61L 15/40; A61L 15/60; A61L 15/48; A61L 15/24; A61F 2013/15463; A61F 2013/530021; A61F 2013/530014

(57) ABSTRACT

Absorbent fibrous structures containing a branched copolymer soil adsorbing agent and methods for making same are provided.

### FIELD OF THE INVENTION

The present invention relates to absorbent fibrous structures and more particularly to absorbent fibrous structures comprising a branched copolymer soil adsorbing agent and methods for making same.

### BACKGROUND OF THE INVENTION

Fibrous structures comprising a soil adsorbing agent are known in the art. Oftentimes with such fibrous structures, the soil adsorbing agents are applied to one or more surfaces of the fibrous structure rather than the soil adsorbing agents being added to during the fibrous structure making process, for example to a fiber slurry used to make the fibrous structure. However, fibrous structures comprising soil adsorbing agents that have been added in the fibrous structure making process in the form of an aqueous solution of a linear soil adsorbing polymer and/or as an emulsion, inverted or not, of a branched soil adsorbing polymer, such as in a fiber slurry used to make the fibrous structure, have negatively impacted the absorption properties, for example the absorptive rate, such as the CRT Initial Rate of the fibrous structures.

One problem with current fibrous structures comprising a soil adsorbing agent that has been added during the fibrous structure making process is that the soil adsorbing agent negatively impacts the absorption properties, for example absorption rate (CRT Initial Rate) as measured according to the CRT Test Method described herein, of the fibrous structures.

Accordingly, there is a need for a fibrous structure comprising a soil adsorbing agent that has been added during the fibrous structure making process that doesn't exhibit the negatives described above; namely, doesn't exhibit the absorption property negatives that current soil adsorbing agents exhibit and a method for making such fibrous structures.

### SUMMARY OF THE INVENTION

The present invention fulfills the needs described above by providing a fibrous structure comprising a branched copolymer soil adsorbing agent that overcomes the negatives described above.

One solution to the problem identified above is a fibrous structure comprising a branched copolymer soil adsorbing agent, for example at a lower level less than 7#/ton and/or less than 5#/ton to greater than 0.5#/ton and/or greater than 1#/ton and/or greater than 2#/ton) of the branched copolymer soil adsorbing agent, and a process for making a fibrous structure that adds a branched copolymer at the wet-end of the fibrous structure making, for example papermaking process, such as in the fiber slurry.

In one example of the present invention, an absorbent fibrous structure comprising a branched copolymer soil adsorbing agent randomly dispersed throughout the fibrous structure, for example a non-surface applied branched copolymer soil adsorbing agent, such that the absorbent fibrous structure exhibits a CRT Initial Rate greater than the CRT Initial Rate as measured according to the CRT Test Method described herein of the fibrous structure void of soil adsorbing agents, is provided.

In another example of the present invention, an absorbent fibrous structure comprising a branched copolymer soil adsorbing agent randomly dispersed throughout the fibrous structure, for example a non-surface applied branched copolymer soil adsorbing agent, such that the absorbent fibrous structure exhibits a CRT Initial Rate of greater than 0.54 g/second and/or greater than 0.55 g/second and/or greater than 0.57 g/second and/or greater than 0.60 g/second and/or greater than 0.65 g/second and/or greater than 0.70 g/second and/or about 0.72 g/second as measured according to the CRT Test Method described herein, is provided.

In still another example of the present invention, a single- or multi-ply sanitary tissue product comprising an absorbent fibrous structure according to the present invention, is provided.

In yet another example of the present invention, a method for making an absorbent fibrous structure comprising the steps of:
a. providing a fiber slurry, such as an aqueous fiber slurry, for example comprising a plurality of fibers, such as wood pulp fibers;
b. adding a branched copolymer soil adsorbing agent to the fiber slurry; and
c. forming a fibrous structure from the fiber slurry such that the fibrous structure exhibits a CRT Initial Rate of greater than the fibrous structure void of soil adsorbing agents, is provided.

In yet another example of the present invention, a method for making an absorbent fibrous structure comprising the steps of:
a. providing a fiber slurry, such as an aqueous fiber slurry, for example comprising a plurality of fibers, such as wood pulp fibers;
b. adding a branched copolymer soil adsorbing agent to the fiber slurry; and
c. forming a fibrous structure from the fiber slurry such that the fibrous structure exhibits a CRT Initial Rate of greater than 0.54 g/second and/or greater than 0.55 g/second and/or greater than 0.57 g/second and/or greater than 0.60 g/second and/or greater than 0.65 g/second and/or greater than 0.70 g/second and/or about 0.72 g/second as measured according to the CRT Test Method described herein, is provided.

The present invention provides novel absorbent fibrous structures comprising a branched copolymer soil adsorbing agent adsorbing agent, and methods for making same.

### DETAILED DESCRIPTION OF THE INVENTION

### Definitions

“Fibrous structure” as used herein means a structure that comprises one or more fibrous filaments and/or fibers. In one example, a fibrous structure according to the present invention means an orderly arrangement of filaments and/or fibers within a structure in order to perform a function. Non-limiting examples of fibrous structures of the present invention include paper, fabrics (including woven, knitted, and non-woven), and absorbent pads (for example for diapers or feminine hygiene products).

Non-limiting examples of processes for making fibrous structures include known wet-laid processes, such as wet-laid papermaking processes, and air-laid processes, such as air-laid papermaking processes. Wet-laid and/or air-laid papermaking processes typically include a step of preparing a composition comprising a plurality of fibers that are suspended in a medium, either wet, more specifically aqueous medium, or dry, more specifically gaseous medium, such as air. The aqueous medium used for wet-laid processes is oftentimes referred to as a fiber slurry. The fiber composition is then used to deposit a plurality of fibers onto a forming wire or belt such that an embryonic fibrous structure is formed, after which drying and/or bonding the fibers together results in a fibrous structure. Further processing the fibrous structure may be carried out such that a finished fibrous structure is formed. For example, in typical papermaking processes, the finished fibrous structure is the fibrous structure that is wound on the reel at the end of papermaking, and may subsequently be converted into a finished product, e.g. a sanitary tissue product.

Non-limiting examples of other known processes and/or unit operations for making fibrous structures include fabric crepe and/or belt crepe processes, ATMOS processes, NTT processes, through-air-dried processes, uncreped through-air-dried processes, and conventional wet press processes.

Another process that can be used to produce the fibrous structures is a melt-blowing, dry spinning, and/or spunbonding process where a polymer composition is spun into filaments and collected on a belt to produce a fibrous structure. In one example, a plurality of fibers may be mixed with the filaments prior to collecting on the belt and/or a plurality of fibers may be deposited on a prior produced fibrous structure comprising filaments.

The fibrous structures of the present invention may be homogeneous or may be layered in the direction normal to the machine direction. If layered, the fibrous structures may comprise at least two and/or at least three and/or at least four and/or at least five layers.

The fibrous structures of the present invention may be co-formed fibrous structures. “Co-formed” as used herein means that the fibrous structure comprises a mixture of at least two different components wherein at least one of the components comprises a filament, such as a polypropylene filament, and at least one other component, different from the first component, comprises a solid additive, such as a fiber and/or a particulate. In one example, a co-formed fibrous structure comprises solid additives, such as fibers, such as wood pulp fibers and/or absorbent gel articles of manufacture and/or filler particles and/or particulate spot bonding powders and/or clays, and filaments, such as polypropylene filaments.

“Solid additive” as used herein means a fiber and/or a particulate.

“Particulate” as used herein means a granular substance or powder.

“Fiber” and/or “Filament” as used herein means an elongate particulate having an apparent length greatly exceeding its apparent width, i.e. a length to diameter ratio of at least about 10. In one example, a “fiber” is an elongate particulate as described above that exhibits a length of less than 5.08 cm (2 in.) and a “filament” is an elongate particulate as described above that exhibits a length of greater than or equal to 5.08 cm (2 in.).

Fibers are typically considered discontinuous in nature. Non-limiting examples of fibers include wood pulp fibers and synthetic staple fibers such as polyester fibers.

Filaments are typically considered continuous or substantially continuous in nature. Filaments are relatively longer than fibers. Non-limiting examples of filaments include meltblown and/or spunbond filaments. Non-limiting examples of articles of manufacture that can be spun into filaments include natural polymers, such as starch, starch derivatives, cellulose and cellulose derivatives, hemicellulose, hemicellulose derivatives, and synthetic polymers including, but not limited to polyvinyl alcohol filaments and/or polyvinyl alcohol derivative filaments, and thermoplastic polymer filaments, such as polyesters, nylons, polyolefins such as polypropylene filaments, polyethylene filaments, and biodegradable or compostable thermoplastic fibers such as polylactic acid filaments, polyhydroxyalkanoate filaments and polycaprolactone filaments. The filaments may be monocomponent or multicomponent, such as bicomponent filaments.

In one example of the present invention, “fiber” refers to papermaking fibers. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. Also applicable to the present invention are fibers derived from recycled paper, which may contain any or all of the above categories as well as other non-fibrous articles of manufacture such as fillers and adhesives used to facilitate the original papermaking.

In addition to the various wood pulp fibers, other cellulosic fibers such as cotton linters, rayon, lyocell and bagasse can be used in this invention. Other sources of cellulose in the form of fibers or capable of being spun into fibers include grasses and grain sources.

“Absorbent fibrous structure” as used herein means a fibrous structure that absorbs water. “Dry web” as used herein means a web that comprises less than 30% and/or less than 20% and/or less than 15% and/or less than 10% and/or less than 7% and/or less than 5% and/or less than 3% and/or less than 2% and/or less than 1% and/or less than 0.5% by weight of moisture as measured according to the Moisture Content Test Method described herein.

“Dry absorbent fibrous structure” as used herein means an absorbent fibrous structure that comprises less than 30% and/or less than 20% and/or less than 15% and/or less than 10% and/or less than 7% and/or less than 5% and/or less than 3% and/or less than 2% and/or less than 1% and/or less than 0.5% by weight of moisture as measured according to the Moisture Content Test Method described herein.

“Sanitary tissue product” as used herein means a soft, low density (i.e. <about 0.15 g/cm³) web useful as a wiping implement for post-urinary and post-bowel movement cleaning (toilet tissue), for otorhinolaryngological discharges (facial tissue), multi-functional absorbent and cleaning uses (absorbent towels), and folded sanitary tissue products such as napkins and/or facial tissues including folded sanitary tissue products dispensed from a container, such as a box. The sanitary tissue product may be convolutedly wound upon itself about a core or without a core to form a sanitary tissue product roll.

In one example, the sanitary tissue product of the present invention comprises a fibrous structure according to the present invention.

The sanitary tissue products of the present invention may exhibit a basis weight between about 10 g/m² to about 120 g/m² and/or from about 15 g/m² to about 110 g/m² and/or from about 20 g/m² to about 100 g/m² and/or from about 30 to 90 g/m² as measured according to the Basis Weight Test Method described herein In addition, the sanitary tissue product of the present invention may exhibit a basis weight between about 40 g/m² to about 120 g/m² and/or from about 50 g/m² to about 110 g/m² and/or from about 55 g/m² to about 105 g/m² and/or from about 60 to 100 g/m² as measured according to the Basis Weight Test Method described herein.

The sanitary tissue products of the present invention may be in the form of sanitary tissue product rolls. Such sanitary tissue product rolls may comprise a plurality of connected, but perforated sheets of fibrous structure, that are separably dispensable from adjacent sheets. In one example, one or more ends of the roll of sanitary tissue product may comprise an adhesive and/or dry strength agent to mitigate the loss of fibers, especially wood pulp fibers from the ends of the roll of sanitary tissue product.

The sanitary tissue products of the present invention may comprise additives such as softening agents, temporary wet strength agents, permanent wet strength agents, bulk softening agents, lotions, silicones, wetting agents, latexes, especially surface-pattern-applied latexes, dry strength agents such as carboxymethylcellulose and starch, and absorbency aids.

“Basis Weight” as used herein is the weight per unit area of a sample reported in lbs/3000 ft² or g/m² and is measured according to the Basis Weight Test Method described herein.

“By weight of moisture” or “moisture content” means the amount of moisture present in an article of manufacture measured according to the Moisture Content Test Method described herein immediately after the article of manufacture has been conditioned in a conditioned room at a temperature of 73° F.±4° F. (about 23° C.±2.2° C.) and a relative humidity of 50%±10% for 2 hours.

“Water-soluble” as used herein means a material, such as a polymer, for example a soil adsorbing polymer that is miscible in water. In other words, a material that is capable of forming a stable (does not separate for greater than 5 minutes after forming the homogeneous solution) homogeneous solution with water at ambient conditions (about 23° C. and a relative humidity of about 50%).

“Machine Direction” or “MD” as used herein means the direction parallel to the flow of the fibrous structure through the fibrous structure making machine and/or sanitary tissue product manufacturing equipment.

“Cross Machine Direction” or “CD” as used herein means the direction parallel to the width of the fibrous structure making machine and/or sanitary tissue product manufacturing equipment and perpendicular to the machine direction.

“Ply” as used herein means an individual, integral fibrous structure.

“Plies” as used herein means two or more individual, integral fibrous structures disposed in a substantially contiguous, face-to-face relationship with one another, forming a multi-ply fibrous structure and/or multi-ply sanitary tissue product. It is also contemplated that an individual, integral fibrous structure can effectively form a multi-ply fibrous structure, for example, by being folded on itself.

### Absorbent Fibrous Structure

In one example of the present invention, the absorbent fibrous structure comprises a branched copolymer soil adsorbing agent.

In one example, the absorbent fibrous structure of the present invention comprises a dry absorbent fibrous structure such as a dry paper towel, rather than a pre-moistened, liquid composition-containing towel or wipe or pad.

In one example, the absorbent fibrous structure of the present invention exhibits an Average Soil Adsorption Value of greater than 90 and/or greater than 95 and/or greater than 100 and/or greater than 110 and/or greater than 125 and/or greater than 150 and/or greater than 175 and/or greater than 200 mg Soil/g of Absorbent Fibrous Structure as measured according to the Soil Adsorption Test Method described herein before (initially) and after being subjected to the Accelerated and Stress Aging Procedures described herein.

It has been unexpectedly found that absorbent fibrous structures comprising a branched copolymer soil adsorbing agent exhibit a CRT Initial Rate of greater than the fibrous structures void of soil adsorbing agents, for example a CRT Initial Rate of greater than 0.54 g/second and/or greater than 0.55 g/second and/or greater than 0.57 g/second and/or greater than 0.60 g/second and/or greater than 0.65 g/second and/or greater than 0.70 g/second and/or about 0.72 g/second as measured according to the CRT Test Method described herein. It has further unexpectedly been found that absorbent fibrous structures comprising a branched copolymer soil adsorbing agent according to the present invention exhibit a CRT Initial Rate Change of greater than 5% and/or greater than 7% and/or greater than 10% and/or greater than 12% and/or less than 20% and/or greater than 25% and/or greater than 30% as measured according to the CRT Test Method described herein.

It has further been unexpectedly found that absorbent fibrous structures comprising a branched copolymer soil adsorbing agent exhibit a CRT Absorptive Capacity of greater than the fibrous structures void of soil adsorbing agents, for example a CRT Absorptive Capacity of greater than 69.5 g/sheet and/or greater than 70 g/sheet and/or greater than 71 g/sheet and/or greater than 72 g/sheet and/or greater than 73 g/sheet and/or greater than 74 g/sheet and/or greater than 75 g/sheet and/or greater than 76 g/sheet as measured according to the CRT Test Method described herein.

The absorbent fibrous structure may be a dry absorbent fibrous structure.

The absorbent fibrous structure of the present invention may comprise a plurality of pulp fibers. Further, the absorbent fibrous structure of the present invention may comprise a single-ply or multi-ply sanitary tissue product, such as a paper towel.

In another example, the absorbent fibrous structure may be in the form of a cleaning pad suitable for use with a cleaning device, such as a floor cleaning device, for example a Swiffer® cleaning pad or equivalent cleaning pads.

In one example, the branched copolymer soil adsorbing agent present in the absorbent fibrous structure may provide a visual signal resulting from an increased concentration of soil adsorbed onto the branched copolymer soil adsorbing agent.

In addition to the branched copolymer soil adsorbing agent the absorbent fibrous structure may comprise other ingredients, for example one or more surfactants. The surfactants may be present in and/or on the absorbent fibrous structure at a level of from about 0.01% to about 0.5% by weight of the absorbent fibrous structure. Non-limiting examples of suitable surfactants include C₈₋₁₆ alkyl polyglucoside, cocoamido propyl sulfobetaine, and mixtures thereof.

In one example, the absorbent fibrous structure comprises a signal, such as a dye and/or pigment that becomes visible or becomes invisible to a consumer's eye when the absorbent fibrous structure adsorbs soil and/or when a soil adsorbing agent present in the absorbent fibrous structure adsorbs soil. In another example, the signal may be a difference in texture of the absorbent fibrous structure or a difference in the physical state of the absorbent fibrous structure, for example the absorbent fibrous structure dissolves and/or vaporizes when the absorbent fibrous structure adsorbs soil.

### Copolymer (Branched) Soil Adsorbing Agent

In one example, the branched copolymer soil adsorbing agent exhibits a weight average molecular weight of greater than 750,000 and/or greater than 1,500,000 and/or greater than 4,000,000 and/or to about 40,000,000 and/or to about 20,000,000 and/or to about 10,000,000.

In another example, the branched copolymer soil adsorbing agent exhibits a number average molecular weight of greater than 200,000 g/mol and/or greater than 500,000 g/mol and/or greater than 750,000 g/mol and/or greater than 900,000 g/mol to less than 2,000,000 g/mol and/or less than 1,750,000 g/mol and/or less than 1,500,000 g/mol. In one example, the soil adsorbing agent exhibits a number average molecular weight of from about 500,000 g/mol to about 2,000,000 g/mol and/or from about 900,000 g/mol to about 1,700,000 g/mol.

In one example, the branched copolymer soil adsorbing agent comprises monomeric units derived from acrylic acid and/or quaternary ammonium compounds, for example acryloyloxyethyltrimethyl ammonium chloride, and/or acrylamide.

In one example, the fibrous structure comprises greater than 0.005% and/or greater than 0.01% and/or greater than 0.05% and/or greater than 0.1% and/or greater than 0.15% and/or to about 5% and/or to about 4% and/or to about 3% and/or to about 2% and/or to about 1.5% and/or to about 1% and/or to about 0.75% and/or to about 0.5% by weight of the fibrous structure of the branched copolymer soil adsorbing agent. In one example, the fibrous structure comprises from about 0.005% to about 5% and/or from about 0.1% to about 3% and/or from about 0.1% to about 1% and/or from about 0.1% to about 0.35% by weight of the fibrous structure of the branched copolymer soil adsorbing agent. In another example, the fibrous structure comprises greater than 0.5#/ton and/or greater than 1#/ton and/or greater than 1.5#/ton and/or greater than 2#/ton and/or greater than 3#/ton and/or to about 10#/ton and/or to about 8#/ton and/or to about 7#/ton and/or to about 6#/ton by weight of the fibrous structure of the branched copolymer soil adsorbing agent.

In one example, the branched copolymer soil adsorbing agents of the present invention are made one or more monomers, such as a nonionic monomer, for example acrylamide, with a cationic monomer, for example 2-(dimethylamino)ethyl methacrylate (DMAM) (for example about 100-1000 moles per 10,000-20,000 moles of acrylamide), in the presence of bismethylene acrylamide (for example about 1-2 moles per 10,000-20,000 moles of acrylamide). The level of branching and hence the level of bismethylene acrylamide per moles of acrylamide (too high results in a crosslinked copolymer) determine whether the resulting copolymer is “branched” or “crosslinked”. For purposes of the present invention, the resulting copolymer should be branched not crosslinked in order for the copolymer to exhibit its desired function of soil adsorbing.

The branched copolymer soil adsorbing agents are cationic under pH 4.5 conditions. In one example, the branched copolymer soil adsorbing agent comprises a quaternary ammonium compound under pH 4.5 conditions. In another example, the branched copolymer soil adsorbing agent comprises an amine under pH 4.5 conditions. In still another example, the branched copolymer soil adsorbing agent comprises an acrylamide under pH 4.5 conditions. In even another example, the branched copolymer soil adsorbing agent comprises an acrylamide monomeric unit and a quaternary ammonium monomeric unit, for example an acryloyloxyethyltrimethyl ammonium chloride monomeric unit, under pH 4.5 conditions.

The branched copolymer soil adsorbing agent may comprise one or more monomeric units derived from quaternary ammonium compounds, amine compounds, acrylamide compounds, acrylic acid compounds and mixtures thereof at various weight ratios within the polymer.

In one example, the branched copolymer soil adsorbing agent is a copolymer of acrylamide and one or more other nonionic monomers, for example non-acrylamide monomers, such as hydroxyalkylacrylate, for example hydroxypropylacrylate.

In another example, the branched copolymer soil adsorbing agent is a copolymer of acrylamide and one or more cationic monomers, for example quaternary ammonium monomers, such as acryloyloxyethyltrimethyl ammonium chloride.

In one example, the branched copolymer soil adsorbing agent is a branched copolymer of acrylamide, one or more quaternary ammonium monomers, and bismethyleneacrylamide, which is a crosslinking agent that converts a typical linear polyacrylamide into a branched structure. The bismethyleneacrylamide may be present in the branched copolymer soil adsorbing agent at a level of less than 200 ppm and/or less than 100 ppm and/or less than 50 ppm and/or greater than 1 ppm and/or greater than 2 ppm and/or greater than 10 ppm and/or greater than 20 ppm. In one example, the bismethyleneacrylamide may be present in the branched copolymer soil adsorbing agent at a level of from about 2.5 ppm to about 25 ppm.

In another example, the branched copolymer soil adsorbing agent is a copolymer of acrylamide and hydroxyalkylacrylate, such as hydroxypropylacrylate. The hydroxyalkylacrylate may be present in the copolymer at a level of less than 50% and/or less than 40% and/or less than 30% and/or less than 20% and/or less than 10% and/or less than 5% and/or greater than 0.01% and/or greater than 0.1% and/or greater than 0.5%. In one example, the hydroxyalkylacrylate may be present in the copolymer at a level of from about 1% to about 3%.

The branched copolymer soil adsorbing agent of the present invention may comprise a nonionic monomeric unit, such as a nonionic monomeric unit derived from an acrylamide compound. Non-limiting examples of suitable nonionic monomeric units include nonionic monomeric units derived from nonionic monomers selected from the group consisting of: hydroxyalkyl esters of α,β-ethylenically unsaturated acids, such as hydroxyethyl or hydroxypropyl acrylates and methacrylates, glyceryl monomethacrylate, α,β-ethylenically unsaturated amides such as acrylamide, N,N-dimethylmethacrylamide, N-methylolacrylamide, α,β-ethylenically unsaturated monomers bearing a water-soluble polyoxyalkylene segment of the poly(ethylene oxide) type, such as poly(ethylene oxide) α-methacrylates (Bisomer S20W, S10W, etc., from Laporte) or α,ω-dimethacrylates, Sipomer BEM from Rhodia (ω-behenyl polyoxyethylene methacrylate), Sipomer SEM-25 from Rhodia (ω-tristyrylphenyl polyoxyethylene methacrylate), α,β-ethylenically unsaturated monomers which are precursors of hydrophilic units or segments, such as vinyl acetate, which, once polymerized, can be hydrolyzed in order to give rise to vinyl alcohol units or polyvinyl alcohol segments, vinylpyrrolidones, α,β-ethylenically unsaturated monomers of the ureido type, and in particular 2-imidazolidinone-ethyl methacrylamide (Sipomer WAM II from Rhodia). Other nonionic monomeric units suitable for the present invention include nonionic monomeric units derived from nonionic monomers selected from the group consisting of: vinylaromatic monomers such as styrene, alpha-methylstyrene, vinyltoluene, vinyl halides or vinylidene halides, such as vinyl chloride, vinylidene chloride, C₁-C₁₂ alkylesters of α,β-monoethylenically unsaturated acids such as methyl, ethyl or butyl acrylates and methacrylates, 2-ethylhexyl acrylate, vinyl esters or allyl esters of saturated carboxylic acids, such as vinyl or allyl acetates, propionates, versatates, stearates, α,β-monoethylenically unsaturated nitriles containing from 3 to 12 carbon atoms, such as acrylonitrile, methacrylonitrile, α-olefins such as ethylene, conjugated dienes, such as butadiene, isoprene, chloroprene.

The branched copolymer soil adsorbing agents of the present invention may comprise an anionic monomeric unit, such as an anionic monomeric unit derived from acrylic acid. Non-limiting examples of anionic monomeric units suitable for the present invention include anionic monomeric units derived from anionic monomers selected from the group consisting of: monomers having at least one carboxylic function, for instance α,β-ethylenically unsaturated carboxylic acids or the corresponding anhydrides, such as acrylic, methacrylic or maleic acids or anhydrides, fumaric acid, itaconic acid, N-methacroylalanine, N-acryloylglycine, and their water-soluble salts, monomers that are precursors of carboxylate functions, such as tert-butyl acrylate, which, after polymerization, give rise to carboxylic functions by hydrolysis, monomers having at least one sulfate or sulfonate function, such as 2-sulfooxyethyl methacrylate, vinylbenzene sulfonic acid, allyl sulfonic acid, 2-acrylamido-2-methylpropane sulfonic acid (AMPS), sulfoethyl acrylate or methacrylate, sulfopropyl acrylate or methacrylate, and their water-soluble salts, monomers having at least one phosphonate or phosphate function, such as vinylphosphonic acid, etc., the esters of ethylenically unsaturated phosphates, such as the phosphates derived from hydroxyethyl methacrylate (Empicryl 6835 from Rhodia) and those derived from polyoxyalkylene methacrylates, and their water-soluble salts, and 2-carboxyethyl acrylate (CEA).

In one example, the soil adsorbing copolymer comprises a nonionic monomeric unit derived from an acrylamide compound and an anionic monomeric unit derived from acrylic acid.

The branched copolymer soil adsorbing agents of the present invention may comprise a cationic monomeric unit, such as a cationic monomeric unit derived from cationic monomers, for example quaternary ammonium compound monomers, selected from the group consisting of:

N,N-(dialkylamino-w-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride (ADAM methyl chloride); 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT) and 2-hydroxy-N¹-(3-(2 ((3-methacrylamidopropyl)dimethylammino)-acetamido)propyl)-N¹, N¹, N³, N³, N³-pentamethylpropane-1,3-diaminium chloride (TRIQUAT), and mixtures thereof.

In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from 2-hydroxy-N¹-(3-(2((3-methacrylamidopropyl)dimethylammino)-acetamido)propyl)-N¹, N¹, N³, N³, N³-pentamethylpropane-1,3-diaminium chloride. In one example, the branched copolymer soil adsorbing agent exhibits a positive charge density as measured according to the Charge Density Test Method, described herein. In another example, the branched copolymer soil adsorbing agent exhibits a net charge density of greater than 0 meq/g and/or greater than 0.5 meq/g and/or greater than 1.0 meq/g and/or greater than 1.5 meq/g to less than 10 meq/g and/or to less than 7 meq/g and/or to less than 5 meq/g and/or from greater than 0 meq/g to about 5.0 meq/g and/or from about 0.5 meq/g to about 4.5 meq/g and/or from about 1.0 meq/g to about 4.0 meq/g as measured according to the Charge Density Test Method, described herein.

In one example, the branched copolymer soil adsorbing agent is present in the absorbent fibrous structure at a level of greater than 0.005% by weight of the absorbent fibrous structure. In another example, the branched copolymer soil adsorbing agent is present in the absorbent fibrous structure at a level of from about 0.005% to about 5% and/or from about 0.005% to about 3% by weight of the absorbent fibrous structure.

### Process for Making Absorbent Fibrous Structure

An absorbent fibrous structure suitable for use in the present invention may be made by any suitable process known in the art.

In one example, a process for making an absorbent fibrous structure, such as a wet-laid fibrous structure, comprising a branched copolymer soil adsorbing agent of the present invention comprises the steps of:
a. providing a fiber slurry;
b. adding a branched copolymer soil adsorbing agent to the fiber slurry;
c. depositing the fiber slurry onto a foraminous wire to form an embryonic web; and
d. drying the embryonic web, for example at least partially on a patterned belt, to produce a fibrous structure such that the fibrous structure exhibits a CRT Initial Rate as measured according to the CRT Test Method described herein that is greater than the fibrous structure void of soil adsorbing agents.

In another example, a process for making an absorbent fibrous structure, such as a wet-laid fibrous structure, comprises the steps of:
a. providing a fiber slurry;
b. adding a branched copolymer soil adsorbing agent to the fiber slurry;
c. depositing the fiber slurry onto a foraminous wire to form an embryonic web; and
d. drying the embryonic web, for example at least partially on a patterned belt, to produce a fibrous structure such that the fibrous structure exhibits a CRT Initial Rate of greater than 0.54 g/second and/or greater than 0.55 g/second and/or greater than 0.57 g/second and/or greater than 0.60 g/second and/or greater than 0.65 g/second and/or greater than 0.70 g/second and/or about 0.72 g/second as measured according to the CRT Test Method described herein.

Table 1 below shows fibrous structures that comprise soil adsorbing agents, both inventive branched copolymer soil adsorbing agents (aqueous solution comprising branched copolymer soil adsorbing agents Hypedloc® CP9260, which is 25% mole cationic, and CP9270, which is 40% mole cationic, both available from Hychem, Inc. of Tampa, Fla.) that were added to the fibrous structures during the fibrous structure making process (into the fiber slurry of the papermaking process) (Samples 1 & 2) and comparative soil adsorbing agents: Sample A—branched copolymer soil adsorbing agent emulsion (Hypedloc® CE7064 available from Hychem, Inc. of Tampa, Fla.) added to surface of fibrous structure and Samples B-F (Hypedloc® CP903, CP908, CP911, CP911H, and CP911HH, respectively, available from Hychem, Inc. of Tampa, Fla.), which were added to the fibrous structures during the fibrous structure making process as aqueous solutions of linear soil adsorbing agents (into the fiber slurry of the papermaking process), and a Control (Bounty® paper towel—2015) and their respective Absorbency Properties.

TABLE 1

| | CRT | CRT Absorptive | Soil Adsorption Value |
|---|---|---|---|
| | Initial Rate | Capacity | mg Soil/g of Absorbent |
| Sample | g/second | g/sheet | Fibrous Structure |
| 1 | 0.723 | 76.5 | 151 |
| 2 | 0.614 | 73.1 | 152 |
| A | 0.427 | 70.8 | 165 |
| B | 0.533 | 70.8 | 150 |
| C | 0.456 | 71.6 | 150 |
| D | 0.465 | 72.8 | 153 |
| E | 0.46 | 72.2 | 161 |
| F | 0.514 | 70.4 | 156 |
| CONTROL | 0.54 | 69.5 | 95.9 |

The absorbent fibrous structures of the present invention may further comprise, in addition to the branched copolymer soil adsorbing agent that is randomly dispersed throughout the fibrous structure, one or more additional soil adsorbing agents, linear or branched, randomly dispersed throughout the fibrous structure and/or present on a surface of the fibrous structure.

The fiber slurries and/or absorbent fibrous structures may comprise permanent and/or temporary wet strength agents such as Kymene® (permanent wet strength) and Hercobond® (temporary wet strength) both available from Ashland Inc. and/or Parez® (wet strength chemistries) available from Kemira Chemicals, Inc.

The fiber slurries and/or absorbent fibrous structures may comprise dry strength agents such as carboxymethylcellulose, starch, polyvinylamides, polyethyleneimines, melamine/formaldehyde, epoxide, and mixtures thereof.

In still yet another example, a process for making an absorbent fibrous structure, such as an air-laid fibrous structure, comprises the steps of:
a. providing pulp fibers;
b. adding a branched copolymer soil adsorbing agent to the pulp fibers to form treated pulp fibers;
c. producing an air-laid fibrous structure from the treated pulp fibers such that the air-laid fibrous structure exhibits a CRT Initial Rate as measured according to the CRT Test Method described herein that is greater than the fibrous structure void of soil adsorbing agents; and
d. optionally applying a binder, for example a latex binder, to a surface of the air-laid fibrous structure.

In still yet another example, a process for making an absorbent fibrous structure, such as an air-laid fibrous structure, comprises the steps of:
a. providing pulp fibers;
b. adding a branched copolymer soil adsorbing agent to the pulp fibers to form treated pulp fibers;
c. producing an air-laid fibrous structure from the treated pulp fibers such that the fibrous structure exhibits a CRT Initial Rate of greater than 0.54 g/second and/or greater than 0.55 g/second and/or greater than 0.57 g/second and/or greater than 0.60 g/second and/or greater than 0.65 g/second and/or greater than 0.70 g/second and/or about 0.72 g/second as measured according to the CRT Test Method described herein; and
d. optionally applying a binder, for example a latex binder, to a surface of the air-laid fibrous structure.

### Non-Limiting Example

An example of an absorbent fibrous structure according to the present invention; namely, a paper towel, is produced utilizing a cellulosic pulp fiber furnish consisting of about 55% refined softwood furnish consisting of about 44% Northern Bleached Softwood Kraft (Bowater), 44% Northern Bleached Softwood Kraft (Celgar) and 12% Southern Bleached Softwood Kraft (Alabama River Softwood, Weyerhaeuser); about 30% of unrefined hardwood Eucalyptus Bleached Kraft consisting of about 80% (Fibria) and 20% NBHK (Aspen) (Peace River); and about 15% of an unrefined furnish consisting of a blend of about 27% Northern Bleached Softwood Kraft (Bowater), 27% Northern Bleached Softwood Kraft (Celgar), 42% Eucalyptus Bleached Kraft (Fibria) and 7% Southern Bleached Kraft (Alabama River Softwood, Weyerhaeuser). The 55% refined softwood is refined as needed to maintain target wet burst at the reel. Any furnish preparation and refining methodology common to the papermaking industry can be utilized.

A 3% active solution Kymene 5221 is added to the refined softwood line prior to an in-line static mixer and 1% active solution of Wickit 1285, an ethoxylated fatty alcohol available from Ashland Inc. is added to the unrefined Eucalyptus Bleached Kraft (Fibria) hardwood furnish. The addition levels are 21 and 1 lbs active/ton of paper, respectively.

The refined softwood and unrefined hardwood and unrefined NBSK/SSK/Eucalyptus bleached kraft/NDHK thick stocks are then blended into a single thick stock line followed by addition of 1% active carboxymethylcellulose (CMC-Finnfix) solution at 7 lbs active/ton of paper towel, and optionally, a softening agent may be added.

The thick stock is then diluted with white water at the inlet of a fan pump to a consistency of about 0.15% based on total weight of softwood, hardwood and simulated broke fiber. The diluted fiber slurry is directed to a non layered configuration headbox such that the wet web formed onto a Fourdrinier wire (foraminous wire). Optionally, a fines retention/drainage aid may be added to the outlet of the fan pump.

Prior to adding the fiber slurry entering the headbox and/or the fiber furnish, a branched copolymer solid adsorbing agent, namely, Hyperfloc® CP9270 is added to the fiber slurry at a level of about 3# active/ton of paper towel.

Dewatering occurs through the Fourdrinier wire and is assisted by deflector and vacuum boxes. The Fourdrinier wire is of a 5-shed, satin weave configuration having 87 machine-direction and 76 cross-direction monofilaments per inch, respectively. The speed of the Fourdrinier wire is about 750 fpm (feet per minute).

The embryonic wet web is transferred from the Fourdrinier wire at a fiber consistency of about 24% at the point of transfer, to a belt, such as a patterned belt through-air-drying resin carrying fabric. In the present case, the speed of the patterned through-air-drying fabric is approximately the same as the speed of the Fourdrinier wire. In another case, the embryonic wet web may be transferred to a patterned belt and/or fabric that is traveling slower, for example about 20% slower than the speed of the Fourdrinier wire (for example a wet molding process). Further de-watering is accomplished by vacuum assisted drainage until the web has a fiber consistency of about 30%.

While remaining in contact with the patterned belt, the web is pre-dried by air blow-through pre-dryers to a fiber consistency of about 65% by weight.

After the pre-dryers, the semi-dry web is transferred to a Yankee dryer and adhered to the surface of the Yankee dryer with a sprayed creping adhesive. The creping adhesive is an aqueous dispersion with the actives consisting of about 75% polyvinyl alcohol, and about 25% CREPETROL® R6390. Optionally a crepe aid consisting of CREPETROL® A3025 may be applied. CREPETROL® R6390 and CREPETROL® A3025 are commercially available from Ashland Inc. (formerly Hercules Inc.). The creping adhesive diluted to about 0.15% adhesive solids and delivered to the Yankee surface at a rate of about 2# adhesive solids based on the dry weight of the web. The fiber consistency is increased to about 97% before the web is dry creped from the Yankee with a doctor blade.

In the present case, the doctor blade has a bevel angle of about 45° and is positioned with respect to the Yankee dryer to provide an impact angle of about 101° and the reel is run at a speed that is about 15% faster than the speed of the Yankee. In another case, the doctor blade may have a bevel angle of about 25° and be positioned with respect to the Yankee dryer to provide an impact angle of about 81° and the reel is run at a speed that is about 10% slower than the speed of the Yankee. The Yankee dryer is operated at a temperature of about 177° C. and a speed of about 800 fpm. The fibrous structure is wound in a roll using a surface driven reel drum having a surface speed of about 656 feet per minute.

The fibrous structure may be subsequently converted into a two-ply paper towel product (an article of manufacture) having a basis weight of about 45 to 54 g/m².

### Test Methods

Unless otherwise specified, all tests described herein including those described under the Definitions section and the following test methods are conducted on samples that have been conditioned in a conditioned room (CTCH room) at a temperature of 23° C.±1.0° C. and a relative humidity of 50%±2% for a minimum of 2 hours prior to the test. All plastic and paper board packaging articles of manufacture must be carefully removed from the paper samples prior to testing. The samples tested are “usable units.” “Usable units” as used herein means sheets, flats from roll stock, pre-converted flats, and/or single or multi-ply products. Except where noted all tests are conducted in such conditioned room, all tests are conducted under the same environmental conditions and in such conditioned room. Any damaged product is discarded. Test samples with defects such as wrinkles, tears, holes, and like are not measured. Samples conditioned as described herein are considered dry samples (such as “dry filaments”) for testing purposes. All instruments are calibrated according to manufacturer's specifications.

### Accelerated and Stress Aging Procedures

Finished Product stability is defined as the ability of the Finished Product to deliver its intended performance after subjection to the normal range of storage, delivery, and retail conditions. Finished product rolls were packaged using 0.6 mil low density polyethylene film (a proprietary film, Extrel EX1560 available from Tredegar Corporation for this limited purpose) following the procedure detailed below:
1. Cut a 2×3 ft section of 0.6 mil low density polyethylene film.
2. Lay two finished product rolls of paper towels on poly film about 4 inches from the edge of the film such that the rolls are aligned with the 3 ft dimension, and fold poly along the length of the poly over top of the length of the rolls.
3. Heat seal the fold using 3 parallel seals ⅓ inch between each parallel line to insure an effective seal along the length of the rolls.
4. Heat seal on one end about an inch from the end of the poly. This forms a “sock” around the two rolls.
5. Taking care to minimize the volume of air that remains within the finished package, heat seal the final end an inch from the final edge of the 3 ft length of poly forming an airtight seal around the rolls.

The relatively long tail on the package permits samples to be taken off the rolls for testing, resealed and returned to the CTCH room for additional aging. Accelerated and Stress aging conditions are as follows:

Accelerated Aging (40° C.+/−2°, 75% RH+/−5% for 3 months);

Stress Aging (50° C.+/−2°, 60% RH+/−5% for 2 weeks, optionally extended to 3 weeks);

Samples are taken for testing by removing the package from the CTCH room, cutting the end of the package near as possible to the heat seal, remove the rolls, remove 2 sheets from the outside of the rolls and discard, remove 4 full size sheets for mirror cleaning testing and 1 additional sheet for soil retention. Place rolls back into package, and heat seal the top where it was cut and place back into CTCH room for additional aging if necessary. Product aging without packaging under ambient lab conditions (23° C.±1.0° C. and a relative humidity of 50%±2%) has been shown to not occur, therefore test sheets removed from the high CTCH room can be stored under ambient lab conditions without undergoing additional aging before testing.

### Basis Weight Test Method

Basis weight of a fibrous structure, such as sanitary tissue product, is measured on stacks of twelve usable units using a top loading analytical balance with a resolution of ±0.001 g. The balance is protected from air drafts and other disturbances using a draft shield. A precision cutting die, measuring 3.500 in ±0.0035 in by 3.500 in ±0.0035 in is used to prepare all samples.

With a precision cutting die, cut the samples into squares. Combine the cut squares to form a stack twelve samples thick. Measure the mass of the sample stack and record the result to the nearest 0.001 g.

The Basis Weight is calculated in lbs/3000 ft² or g/m² as follows:

Basis Weight=(Mass of stack)/[(Area of 1 square in stack)×(No. of squares in stack)]

For example,

Basis Weight (lbs/3000 ft²)=[[Mass of stack (g)/453.6 (g/lbs)]/[12.25 (in²)/144 (in²/ft²)×12]]×3000

or,

Basis Weight (g/m²)=Mass of stack (g)/[79.032 (cm²)/10,000 (cm²/m²)×12]

Report result to the nearest 0.1 lbs/3000 ft² or 0.1 g/m². Sample dimensions can be changed or varied using a similar precision cutter as mentioned above, so as at least 100 square inches of sample area in stack.

### Moisture Content Test Method

The moisture content present in an article of manufacture, such as a fibrous structure is measured using the following Moisture Content Test Method. An article of manufacture or portion thereof (“sample”) is placed in a conditioned room at a temperature of 23° C.±1.0° C. and a relative humidity of 50%±2% for at least 24 hours prior to testing. Each fibrous structure sample has an area of at least 4 square inches, but small enough in size to fit appropriately on the balance weighing plate. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the sample is placed into a forced air oven on top of foil for 24 hours at 70° C.±2° C. at a relative humidity of 4%±2% for drying. After the 24 hours of drying, the sample is removed and weighed within 15 seconds. This weight is designated as the “dry weight” of the sample.

The moisture content of the sample is calculated as follows:

$$\% Moisture in sample = 100 \% \times \frac{( Equilibrium weight of sample - Dry weight of sample )}{Dry weight of sample}$$

The % Moisture in sample for 3 replicates is averaged to give the reported % Moisture in sample. Report results to the nearest 0.1%.

### Soil Adsorption Test Method

In order to measure an article of manufacture's Average Soil Adsorption Value the following test is conducted.

### Preparation:

A specimen of the article of manufacture, such as a fibrous structure, to be tested is obtained from the central portion of a representative sample of the article of manufacture. The specimen is prepared by cutting a CD strip (extending across the entire CD of the article of manufacture) from an article of manufacture, such as a finished fibrous structure and/or sanitary tissue product sheet (sample) such that the cut CD strip specimen has a length and width resulting in the specimen weighing 0.65 g±0.02 g. The sheet of the sample from which the CD strip specimen is cut may be delineated and connected to adjacent sheets by perforation or tear lines or the sheets of the sample may be individual sheets, such as in the form of individual wipes and/or facial tissues. If connected via perforation or tear lines, then separate one sheet from any adjacent sheet before cutting the CD strip from the sheet. The CD strip specimen needs to be free of perforations and is obtained from a portion of an article of manufacture at least 0.5 inches from any perforations. The specimen is conditioned as described above. The sample weight (W_{Prod}) is recorded to the within ±0.0001 g. A suitable ball-point pen or equivalent marker is used to write the specimen name onto a corner of the specimen.

A centrifuge tube (VWR brand 50 mL superclear ultra high performance freestanding centrifuge tube with flat caps, VWR Catalog #82018-052; or equivalent tube) is labeled with the specimen name and weighed to within ±0.1 mg W_{CT}. Next, 155.0 mg±5.0 mg of a model soil (black todd clay) available from Empirical Manufacturing Co., 7616 Reinhold Drive, Cincinnati, Ohio 45237-3208) is placed into the centrifuge tube. The tube is re-weighed W_{(CT+Soil)} and the model soil weight (W_{Soil}) is determined to nearest 0.2 mg by difference W_{(CT+Soil)}−W_{CT}.

Distilled water, 35 g±0.5 g is added slowly to the centrifuge tube using a suitable dispenser. The centrifuge tube is a VWR brand 50 mL superclear ultra high performance freestanding centrifuge tube with flat caps (VWR Catalog #82018-052, or equivalent tube). The distilled water is poured carefully into the centrifuge tube to avoid causing a plume of dust from the model soil. If a plume of dust occurs such that the weight of soil in the tube may be impacted, the tube is discarded and a new tube is prepared. The tube is then re-weighed W_{(CT+Soil+Water)} and the total weight (W_{(Soil Dispersion)} of water plus soil in the centrifuge tube is calculated by subtracting the weight of the centrifuge tube W_{CT} from the W_{(CT+Soil+Water)} and recorded to the nearest 0.2 mg.

A glass petri dish (e.g. VWR 50×35, VWR Catalog #89000-280, or equivalent dish) is labeled and weighed to within 0.1 mg (W_{(Petri Dish)}).

Testing:

A reciprocating shaker is used to disperse the model soil in the water. The model soil must be completely dispersed for the results to be valid. A reciprocating shaker (IKA Works HS 501 digital reciprocating shaker, number 2527001, with a Universal attachment, number 8000200, or equivalent shaker) is set to 300±3 cycles per minute. The capped centrifuge tube containing the model soil and water is mounted in the shaker and shaken for 30 seconds to obtain a uniform dispersion of the soil in the water (soil dispersion).

The specimen is loosely folded along its transverse centerline with an accordion style (paper fan) folding technique. The specimen is loosely folded 5 times, to produce a sample that contains 10 segments each about 2.5 cm in length. This folding technique keeps the sample from being too tightly folded, which may hinder free flow of water and suspended soil over all surfaces of the article the thus efficiency of the paper to adsorb the soil. The folded sample is fully immersed into the soil dispersion in the centrifuge tube so that the folds run parallel to the length of the centrifuge tube. The tube is immediately re-capped and shaken in the reciprocating shaker for 30+/−1 seconds with the length axis of the centrifuge tube parallel to the motion of the reciprocating shaker.

After shaking, the folded specimen is carefully removed over the glass petri dish using laboratory tweezers. Care must be taken to ensure that greater than 95% of the soil dispersion is kept either in the original centrifuge tube or corresponding glass petri dish. The soil dispersion is wrung (removed) from the specimen using a “wringing” motion and collected in the glass petri dish. Once the soil dispersion has been removed from the specimen, the specimen is discarded. The remaining soil dispersion is poured from the centrifuge tube into the glass petri dish after swirling the mixture to re-disperse model soil into water, thereby ensuring that no model soil is inadvertently left behind in the centrifuge tube. The glass petri dish containing the model soil/water mixture is weighed to within ±0.1 mg W_{(Petri Dish+Soil Dispersion)}. The weight of soil dispersion recovered W_{(Recovered Soil Dispersion)} is calculated by subtracting the weight of the glass vented laboratory drying oven at 105° C. until the sample is residual soil is fully dry. The W_{(Recovered Soil Dispersion)} should be >95% of the W_{(Soil Dispersion)}.

Once the sample is dry, the glass petri dish containing the dried model soil is removed from the oven and placed in a desiccator until cool and then re-weighed to within ±0.1 mg W_{(Petri Dish+Residual Dry Soil)}. The weight of residual soil W_{(Residual Soil)} is calculated by subtracting the weight of the glass petri dish W_{(Petri Dish)} from W_{(Petri Dish+Residual Dry Soil)} and recorded to the nearest 0.2 mg.

### Calculations:

To calculate the amount of residual model soil W_{(Residual Soil)} left in the glass petri dish, the following equation is used:

W_{(Residual Soil)}−W_{(Petri Dish+Residual Dry Soil)}−W_{(Petri Dish)}
Residual model soil weight (W_{(Residual Soil)}) is reported in mg.

To calculate the amount of normalized residual model soil (W_{(Norm Residual Soil)}) left in the glass petri dish, the following equation is used:

W_{(Norm Residual Soil)}=W_{(Residual Soil)}*W_{(Soil Dispersion)}/W_{(Recovered Soil Dispersion)}
Normalized residual soil weight W_{(Norm Residual Soil)} is reported in mg.

To calculate the amount of soil adsorbed by the sample, the following calculation is used:

W_{(Soil Adsorbed)}=(W_{(Soil)}−W_{(Norm Residual Soil)})/W_{(Prod)}
Soil adsorbed in sample W_{(Soil Adsorbed)} is reported as mg soil/g article of manufacture.

The test is performed on three replicates and an Average Soil Adsorption Value (Avg W_{(Soil Adsorbed)}) is calculated for the article of manufacture. These values are measured and calculated for initial Average Soil Adsorption Value of a specimen prior to subjecting the specimen to the Accelerated and Stress Aging Procedures described herein and after subjecting the specimen to the Accelerated and Stress Aging Procedures described herein. Soil Adsorption Value is also referred to herein as mg Soil Retained/gram Paper and its corresponding % Soil Retained (by Paper).

### Charge Density Test Method

If one has identified or knows the soil adsorbing agent in and/or on an article of manufacture, then the charge density of the soil adsorbing agent can be determined by using a Mutek PCD-04 Particle Charge Detector available from BTG, or equivalent instrument. The following guidelines provided by BTG are used. Clearly, manufacturers of articles of manufacture comprising soil adsorbing agents know what soil adsorbing agent(s) are being included in their articles of manufacture. Therefore, such manufacturers and/or suppliers of the soil adsorbing agents used in the articles of manufacture can determine the charge density of the soil adsorbing agent.

1. Start with a 0.1% solution (0.1 g soil adsorbing agent+99.9 g deionized water). Preparation of dilute aqueous solutions in deionized water from inverse or dewatered inverse emulsions are performed as instructed by the supplier of the emulsions and is well known to one of ordinary skill in the art. Depending on the titrant consumption increase or decrease soil adsorbing agent content. Solution pH is adjusted prior to final dilution as charge density of many additives is dependent upon solution pH. A pH of 4.5 is used here for cationic polymers and between 6-7 for anionic polymers. No pH adjustment was necessary for the anionic polymers included in this study.

2. Place 20 mL of sample in the PCD measuring cell and insert piston.

3. Put the measuring cell with piston and sample in the PCD, the electrodes are facing the rear. Slide the cell along the guide until it touches the rear.

4. Pull piston upwards and turn it counter-clock-wise to lock the piston in place.

5. Switch on the motor. The streaming potential is shown on the touch panel. Wait 2 minutes until the signal is stable.

6. Use an oppositely charged titrant (for example for a cationic sample having a positive streaming potential: use an anionic titrant). Titrants are available from BTG consisting of 0.001N PVSK or 0.001N PolyDADMAC.

7. An automatic titrator available from BTG is utilized. After selecting the proper titrant, set the titrator to rinse the tubing by dispensing 10 mL insuring that all air bubbles have been purged.

8. Place tubing tip below the surface of the sample and start titration. The automatic titrator is set to stop automatically when the potential reaches 0 mV.

9. Record consumption of titrant, ideally, the consumption of titrant should be 0.2 mL to 10 mL; otherwise decrease or increase soil adsorbing agent content.

10. Repeat titration of a second 20 mL aliquot of the soil adsorbing agent sample.

11. Calculate charge demand (solution) or charge demand (solids);

$$Charge demand ( eq \text{/} L ) = \frac{V titrant used ( L ) \times Conc . of titrant in Normality ( eq \text{/} L )}{Volume of sample titrated ( L )}$$

$$Charge demand ( eq \text{/} g ) = \frac{V titrant used ( L ) \times Conc . of titrant in Normality ( eq \text{/} L )}{Wt . solids of the sample or its active substance ( g )}$$

The charge density (charge demand) of a soil adsorbing agent is reported in meq/g units.

### CRT Test Method

The absorption (wicking) of water by an absorbent fibrous structure (sample) is measured over time. A sample is placed horizontally in the instrument and is supported by an open weave net structure that rests on a balance. The test is initiated when a tube connected to a water reservoir is raised and the meniscus makes contact with the center of the sample from beneath, at a small negative pressure. Absorption is allowed to occur for 2 seconds after which the contact is broken and the cumulative rate for the first 2 seconds is calculated.

### Apparatus

Conditioned Room—Temperature is controlled from 73° F.+2° F. (23° C.+1° C.). Relative

Humidity is controlled from 50%+2%

Sample Preparation—Product samples are cut using hydraulic/pneumatic precision cutter into 3.375 inch diameter circles.

Capacity Rate Tester (CRT)—The CRT is an absorbency tester capable of measuring capacity and rate. The CRT consists of a balance (0.001 g), on which rests on a woven grid (using nylon monofilament line having a 0.014″ diameter) placed over a small reservoir with a delivery tube in the center. This reservoir is filled by the action of solenoid valves, which help to connect the sample supply reservoir to an intermediate reservoir, the water level of which is monitored by an optical sensor. The CRT is run with a −2 mm water column, controlled by adjusting the height of water in the supply reservoir.

Software—LabView based custom software specific to CRT Version 4.2 or later.

Water—Distilled water with conductivity <10 μS/cm (target <5 μS/cm) @ 25° C. For this method, a usable unit is described as one finished product unit regardless of the number of plies. Condition all samples with packaging materials removed for a minimum of 2 hours prior to testing. Discard at least the first ten usable units from the roll. Remove two usable units and cut one 3.375-inch circular sample from the center of each usable unit for a total of 2 replicates for each test result. Do not test samples with defects such as wrinkles, tears, holes, etc. Replace with another usable unit which is free of such defects

### Pre-Test Set-Up

1. The water height in the reservoir tank is set −2.0 mm below the top of the support rack (where the sample will be placed).
2. The supply tube (8 mm I.D.) is centered with respect to the support net.
3. Test samples are cut into circles of 3⅜″ diameter and equilibrated at Tappi environment conditions for a minimum of 2 hours.

### Test Description

1. After pressing the start button on the software application, the supply tube moves to 0.33 mm below the water height in the reserve tank. This creates a small meniscus of water above the supply tube to ensure test initiation. A valve between the tank and the supply tube closes, and the scale is zeroed.
2. The software prompts you to “load a sample”. A sample is placed on the support net, centering it over the supply tube, and with the side facing the outside of the roll placed downward.
3. Close the balance windows, and press the “OK” button—the software records the dry weight of the circle.
4. The software prompts you to “place cover on sample”. The plastic cover is placed on top of the sample, on top of the support net. The plastic cover has a center pin (which is flush with the outside rim) to ensure that the sample is in the proper position to establish hydraulic connection. Four other pins, 1 mm shorter in depth, are positioned 1.25-1.5 inches radially away from the center pin to ensure the sample is flat during the test. The sample cover rim should not contact the sheet. Close the top balance window and click “OK”.
5. The software re-zeroes the scale and then moves the supply tube towards the sample. When the supply tube reaches its destination, which is 0.33 mm below the support net, the valve opens (i.e., the valve between the reserve tank and the supply tube), and hydraulic connection is established between the supply tube and the sample. Data acquisition occurs at a rate of 5 Hz, and is started about 0.4 seconds before water contacts the sample.
6. The test runs for 2 seconds. After this, the supply tube pulls away from the sample to break the hydraulic connection.
7. The wet sample is removed from the support net. Residual water on the support net and cover are dried with a paper towel.
8. Repeat until all samples are tested.
9. After each test is run, a *.txt file is created (typically stored in the CRT/data/rate directory) with a file name as typed at the start of the test. The file contains all the test set-up parameters, dry sample weight, and cumulative water absorbed (g) vs. time (sec) data collected from the test.
10. Report the average cumulative 0-2 seconds rate to the nearest 0.001 g/second as the CRT Initial Rate.
11. The difference between a Control Sample and a Test Sample can be calculated from their respective CRT Initial Rates from Step 10 and then the percentage change can be determined and reported as CRT Initial Rate Change.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent fibrous structure comprising a branched copolymer soil adsorbing agent such that the absorbent fibrous structure exhibits a CRT Initial Rate that is greater than the CRT Initial Rate of the absorbent fibrous structure void of soil adsorbing agents as measured according to the CRT Test Method.

2. The absorbent fibrous structure according to claim 1 wherein the absorbent fibrous structure exhibits an average Soil Adsorption Value of about 90 mg soil/g absorbent fibrous structure or greater as measured according to the Soil Adsorption Test Method.

3. The absorbent fibrous structure according to claim 1 wherein the absorbent fibrous structure comprises a plurality of pulp fibers.

4. The absorbent fibrous structure according to claim 1 wherein the absorbent fibrous structure exhibits a moisture level of less than 30% as measured according to the Moisture Content Test Method.

5. The absorbent fibrous structure according to claim 1 wherein branched copolymer soil adsorbing agent comprises a monomeric unit derived from an acrylamide compound.

6. The absorbent fibrous structure according to claim 1 wherein the branched copolymer soil adsorbing agent is present in the absorbent fibrous structure at a level of from about 0.005% to about 5% by weight of the absorbent fibrous structure.

7. The absorbent fibrous structure according to claim 1 wherein the absorbent fibrous structure further comprises a surfactant.

8. The absorbent fibrous structure according to claim 1 wherein the absorbent fibrous structure exhibits a CRT Initial Rate of greater than 0.54 g/second as measured according to the CRT Test Method.

9. A single- or multi-ply sanitary tissue product comprising the absorbent fibrous structure according to claim 1.

10. The sanitary tissue product according to claim 10 wherein the sanitary tissue product comprises a paper towel.

11. A cleaning pad comprising the absorbent fibrous structure according to claim 1.

12. An absorbent fibrous structure comprising a branched copolymer soil adsorbing agent such that the absorbent fibrous structure exhibits a CRT Initial Rate that is greater than 0.54 g/second as measured according to the CRT Test Method.

13. The absorbent fibrous structure according to claim 12 wherein the absorbent fibrous structure comprises a plurality of pulp fibers.

14. The absorbent fibrous structure according to claim 12 wherein the absorbent fibrous structure exhibits a moisture level of less than 30% as measured according to the Moisture Content Test Method.

15. The absorbent fibrous structure according to claim 12 wherein branched copolymer soil adsorbing agent comprises a monomeric unit derived from an acrylamide compound.

16. The absorbent fibrous structure according to claim 12 wherein the branched copolymer soil adsorbing agent is present in the absorbent fibrous structure at a level of from about 0.005% to about 5% by weight of the absorbent fibrous structure.

17. The absorbent fibrous structure according to claim 12 wherein the absorbent fibrous structure further comprises a surfactant.

18. A single- or multi-ply sanitary tissue product comprising the absorbent fibrous structure according to claim 12.

19. The sanitary tissue product according to claim 18 wherein the sanitary tissue product comprises a paper towel.

20. A cleaning pad comprising the absorbent fibrous structure according to claim 12.
